(19) United States
(12) Patent Application Publication
(10) Pub. No.: US 2024/0074685 A1
(43) Pub. Date: Mar. 7, 2024

(54) SYSTEMS AND METHODS FOR REAL TIME NONINVASIVE URINE OUTPUT ASSESSMENT

(71) Applicants: THORMED INNOVATION LLC, Dallas, TX (US); Board of Regents, The University of Texas System, Austin, TX (US)

(72) Inventors: Gregory Robert Thoreson, Dallas, TX (US); Kenneth Hoyt, Dallas, TX (US); Katherine G. Brown, Coppell, TX (US)

(21) Appl. No.: 18/271,694

(22) PCT Filed: Feb. 2, 2022
(86) PCT No.: PCT/US2022/014857

§ 371 Date: Jul. 11, 2023

Related U.S. Application Data
(60) Provisional application No. 63/144,484, filed on Feb. 2, 2021.

(51) Int. Cl. A61B 5/20; A61B 8/00; A61B 8/08

(52) U.S. Cl. CPC A61B 5/204; A61B 8/085; A61B 8/4227; A61B 8/4281; A61B 8/4472; A61B 8/4488; A61B 8/462

(57) ABSTRACT

The present disclosure discloses systems and methods for measuring the bladder volume of a patient. An ultrasound transducer array may be used to transmit electrical pulses and receive the backscattered pulses that can be analyzed to develop a representation of the patients bladder. The backscattered ultrasound signals may be amplified, converted, and processed by circuitry and computer software to develop an axial position of the anterior and posterior bladder walls. Bladder volume measurements derived from the patients bladder may then be wirelessly transmitted to a display device for assessment by a healthcare provider. The ultrasound transducer array portion of the device may be held in place between the patients peritoneum and pubis by an elastic strap or a belt to ensure proper positioning.

### PRIORITY CLAIM

This application claims priority to and is filed as a parent of U.S. provisional patent application Ser. No. 63/144,484 that was filed on Feb. 2, 2021. Further, the contents of U.S. provisional patent application Ser. No. 63/144,484 are fully incorporated in this application.

### TECHNICAL FIELD

The present disclosure relates generally to systems and devices that assist with the assessment of urine output for a patient, and more specifically, to devices that will enable healthcare providers to monitor and track urine collection and output in real time without the need to insert a urethral catheter into the patient.

### BACKGROUND

Urethral catheters have been used throughout the history of medicine to measure urine output in critically ill patients and for perioperative use. In fact, the Centers for Disease Control and Prevention (CDC) identifies urethral catheters as the standard of care for measuring urine output for these patients. However, there are significant risks inherent with the use of urethral catheters, including catheter associated urinary tract infections (CAUTIs) and iatrogenic urethral trauma. And urethral catheters are the leading cause of secondary nosocomial blood stream infections, with an associated mortality rate of 10% and a financial impact of $1.82 billion annually in the United States. The invasive use of urethral catheters in critically ill patients or patients going through operative procedures can lead to infection and trauma.

In 2010, indwelling urethral catheters were used during 48.3 million surgical procedures, and in 2013, indwelling urethral catheters were used for over 18 million urinary catheter days for patients admitted to critical care, oncology, and inpatient wards. Indwelling urethral catheters are also used for accurate urine output measurements in patients with acute kidney injury, hypovolemia, hypervolemia, congestive heart failure, oliguria, and electrolyte abnormalities, which accounted for over 21 million hospital admissions and 23.5 million emergency room visits in 2016. Use of urethral catheters by healthcare providers in these situations creates unnecessary risk for patients that are critically ill and are going through operative procedures. Noninvasive options for measuring urine output are desired to limit the risk to these vulnerable patients. Due to the inherent features of the human bladder, urine volume measurements may be made by observing the size of the bladder as it fills with and expels urine. Similar to a balloon filling with air or water, the larger the bladder gets, the more volume of urine inside the bladder.

### BRIEF SUMMARY

The present disclosure includes novel medical devices for use in monitoring and measuring a bladder volume for patients or users by continuous noninvasive assessment of the maximal diameter of a bladder throughout the emptying and filling phases of the micturition cycle. In some embodiments, this bladder volume measurement device may include a sparse ultrasound transducer array for beamsteering and accurate bladder diameter measurement, microprocessor control, wireless connectivity between the main sensing unit and a user display, and data integration with existing patient monitoring systems.

In one embodiment of the present disclosure, an ultrasound transducer array is placed in the suprapubic area of the patient (in between the peritoneum and the pubis). Electrical pulses may be generated, focused, and phased for transmission towards the bladder of the patient by the ultrasound transducer array. The ultrasound transducer array also receives the backscattered ultrasound signals from the patient to develop a representation of the bladder. In some embodiments, the backscattered ultrasound signals enable computer software to determine an axial position of the anterior bladder wall and the posterior bladder wall. Bladder volume may be determined from these representations of the bladder.

In some embodiments of the present disclosure, the backscattered ultrasound signals detected by the ultrasound transducer array may be amplified (linear amplifier), subject to time gain compensation (time gain compensation amplifier), and then converted from analog to digital (A/D converter) by corresponding circuitry and computer software. When these resultant signals are processed with the initial beamsteering data of the generated ultrasound signals, bladder volume measurements may be obtained. After signal processing, the resultant bladder volume measurements may be transmitted wirelessly to a display device for access by a user. From here, healthcare providers may analyze the urine output measurements and assess renal function of the patient.

In some embodiments, an elastic strap or a belt may be used to hold the bladder measurement device in the proper location for accurate measurements. A housing for the ultrasound transducer array and the corresponding circuitry may need to be made of a isoechoic material to prevent any distortion from the interface between skin and device. Wireless connectivity of the device will enable the communication of data metrics to a host system that may be running dedicated computer software for proper bladder volume analysis. A battery may be used to generate voltages needed for operation, while also enabling the patient to operate the device without being tethered to a power supply. The device may include lower power modes, controlled activation, transmission of raw data (instead of processed data), and other features to reduce power consumption and improve battery life.

In some embodiments, additional circuitry, sensors, and software may be used to compensate for movements by the patient, suboptimal placing of the bladder volume measurement device on the patient, poor images of the bladder, and variations in bladder shape and size from patient to patient. As more data is generated by the bladder volume measurement devices in the field, the corresponding software becomes more accurate at calculating the bladder volume measurements.

### BRIEF DESCRIPTION OF THE DRAWINGS

For a more complete understanding of the present disclosure, reference is now made to the following descriptions taken in conjunction with the accompanying drawing, in which:

FIG. 1 shows a side view of a bladder volume measurement device being used on a patient according to certain embodiments of the present disclosure;

FIG. 2 shows a graph representing ultrasound signals that may be used to measure the bladder volume for a patient according to certain embodiments of the present disclosure;

FIG. 3 shows a diagram illustrating electrical pulsing and controlled timing for beamsteering of an ultrasound phased array transducer unit that may be used to measure the bladder volume for a patient according to certain embodiments of the present disclosure;

FIG. 4 shows an architecture for a bladder volume measurement device according to certain embodiments of the present disclosure; and

FIG. 5 shows an anterior view of a bladder volume measurement device attached to a patient according to certain embodiments of the present disclosure.

### DETAILED DESCRIPTION

One of the goals of the present disclosure is to replace the invasive use of indwelling urethral catheters for hemodynamic monitoring and renal function assessment for critically ill patients and those undergoing surgery. With improved accuracy and ease of use, the present disclosure may be used in operating rooms, intensive care units, or even in outpatient settings for the dynamic assessment of urine volumes. In certain embodiments, this bladder volume measurement device may be installed or attached to the patient by nurses, doctors, or even by the patients with minimal instruction. Enabling placement by a patient or user may allow healthcare providers to remotely track urine output as a continuous variable in real time and remotely understand the physiology behind patient symptoms. Unless otherwise specified, use of the terms “connect,” “engage,” “couple,” “attach,” or any other like term describing an interaction between elements is not meant to limit the interaction to direct interaction between the elements and may also include indirect interaction between the elements described.

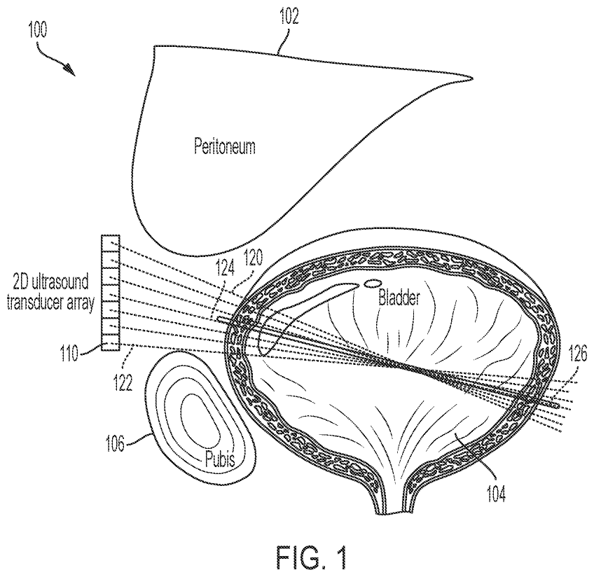

FIG. 1 shows a side view 100 of a bladder volume measurement device being used on a patient according to certain embodiments of the present disclosure. Further details regarding the bladder volume measurement device will be described herein, but an ultrasound transducer array 110 represents a bladder volume measurement device in FIG. 1. The ultrasound transducer array 110 is configured to be placed on the patient in between a peritoneum 102 and a pubis 106 of a patient. The peritoneum 102 is a tissue layer in the abdomen of a patient that is designed to protect the organs, tissues, and blood vessels located in the peritoneum cavity. The pubis 106 is a bone formation that is located in the front of the pelvic girdle of a patient. There is a space in between the peritoneum 102 and the pubis 106 where the ultrasound transducer array 110 of the bladder volume measurement device may be placed to steer signals toward the bladder 104 of a patient.

The ultrasound transducer array 110 may be configured to transmit and receive ultrasound signals. Superior ultrasound signals 120 may be focused on the bladder 104 from a position close to the peritoneum 102. Inferior ultrasound signals 122 may be focused on the bladder 104 from a position close to the pubis 106. Interior ultrasound signals 124 may also be focused on the bladder from around the center of the ultrasound transducer array 110. In combination, these ultrasound signals 120, 122, 124 may be transmitted to the bladder 104 to obtain a view of the features of the bladder 104 by reflecting off these features of the bladder 104 and returning to the ultrasound transducer array 110 for analysis. Backscattered ultrasound signals 126 are designed to bounce off of these features of the bladder 104 and be processed for a representative view of the bladder 104.

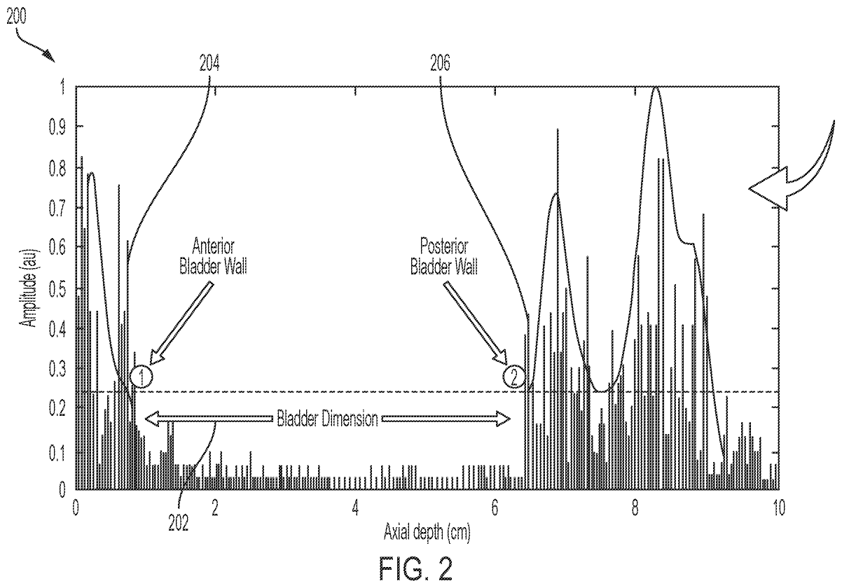

FIG. 2 shows a graph representing ultrasound signals 200 that may be used to measure the bladder volume for a patient according to certain embodiments of the present disclosure. Since the bladder volume (and corresponding diameter) represents the urine inside of the bladder, measuring the bladder size can be used to accurately determine the volume of urine inside the bladder. As shown in FIG. 1, the ultrasound transducer array 110 transmits ultrasound signals toward the patient's bladder 104 and then receives the reflective signals. By adjusting the amplitude, phase, and time delays of the ultrasound signals and then processing the reflective signals, a graph showing the details and features of the bladder 104 can be prepared and analyzed. For example, amplitude may represent the vertical axis and axial depth may represent the horizontal axis in the graph 200. An anterior bladder wall 204 and a posterior bladder wall 206 may be identified by the difference in amplitude in the graph. When the anterior bladder wall 204 and the posterior bladder wall 206 are identified, a bladder dimension 202 may be determined for the patient.

By processing numerous ultrasound signals, the bladder volume device can outline the shape and dimensions of the patient's bladder 104, including shapes and dimensions for the bladder at various stages of fullness. Different patients may have different bladder shapes and sizes, so the bladder volume device may need to initially identify the dimensions of the patient's bladder 104. Once the dimensions are determined, the bladder volume device can track the changes of the bladder to dynamically determine the corresponding volume. More specifically, the changes between the anterior bladder wall 204 and the posterior bladder wall 206 and the corresponding contours of the bladder 104 may be used to calculate the volume and change of volume within the bladder 104.

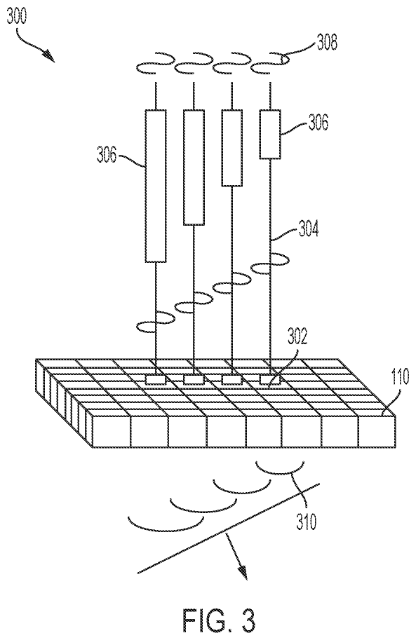

FIG. 3 shows a diagram 300 illustrating electrical pulsing and controlled timing for beamsteering of an ultrasound phased array transducer unit 110 that may be used to measure the bladder volume for a patient according to certain embodiments of the present disclosure. Electrical signals 308 may be generated and transmitted to be sent through an ultrasound transducer array 110. Time delays 306 may be applied to the electrical pulses 308 to improve the ultrasound readings from the patient's bladder 104. These time-adjusted electrical pulses may be sent through the various locations 302 on the phase array transducer unit 110 to create a steered wavefront of signals 310 to transmit to the patient's bladder 104. Thus, depending upon location 302, the electrical pulses create a wavefront 310 upon transmission to the patient's bladder. 104. As these ultrasound electrical pulses 310 reflect from the patient's bladder, the signals can be received and processed to produce a graph such as that in FIG. 2.

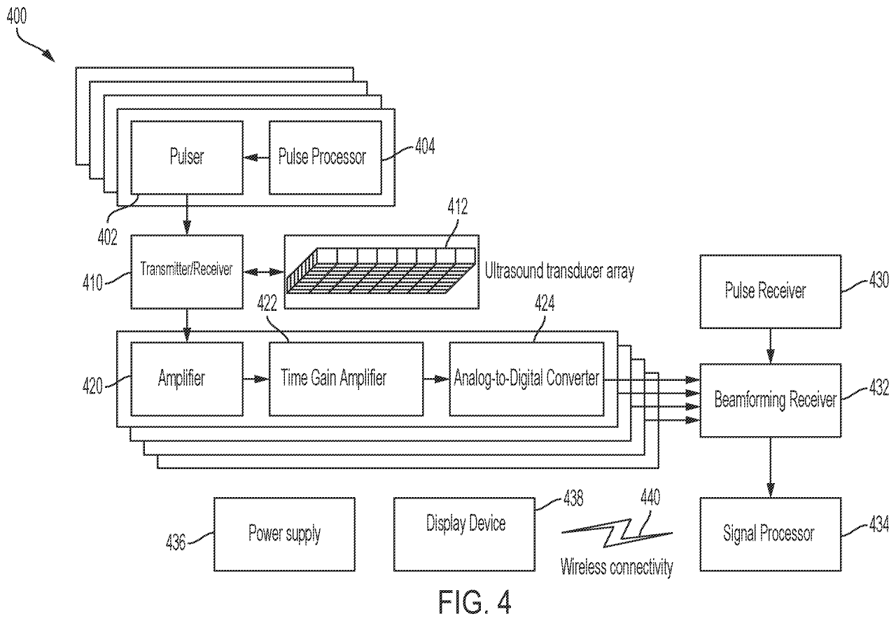

FIG. 4 shows an architecture diagram 400 for a bladder volume measurement device according to certain embodiments of the present disclosure. While this block architecture diagram 400 illustrates a number of components connected in a specific configuration, the present disclosure is not limited to this configuration or the list of components in FIG. 4. Many different configurations are within the scope of the present disclosure and FIG. 4 is only provided to illustrate an embodiment of the present disclosure. As shown in FIG. 3, electrical ultrasound pulses may be created with a pulser 402. A pulse processor 404 may be used to control, adjust, and focus the ultrasound pulses to be transmitted to the patient's abdomen and bladder. As shown in FIG. 4, there may be an array of pulsers 402 to apply numerous pulses to the patient's bladder. A transmitter/receiver 410 receives the ultrasound pulses from the pulser 402 and applies them to an ultrasound transducer array 412. As shown in FIGS. 3-4, the ultrasound transducer array 412 transmits pulses to and receives reflective pulses from the patient's bladder. At this point, the electrical ultrasound pulses have been sent to the patient's bladder.

The remaining components are designed to receive and analyze the pulses that are reflected back from the abdomen of the patient. Once the reflective pulses are received back from the patient by the ultrasound transducer array 412 and the transmitter receiver 410, a linear amplifier 420 may be used to amplify the signals received. A time gain compensation amplifier 422 may also be used to amplify the gain of the signals received to account for tissue attenuation. Then an analog-to-digital converter 424 may be applied to transform the analog signals received to digital for processing. Once again, there may be an array of linear amplifiers 422, time gain compensation amplifiers 422, and analog-to-digital converters 424 to account for the array of pulses supplying the ultrasound signals. An array of signals from the analog-to-digital converters 424 are then transmitted to a beamforming receiver 432 for processing.

A pulse receiver 430 is designed to work in conjunction with the pulse processor 404 to send and receive corresponding ultrasound signals for processing. The array of signals from the analog-to-digital converters 424 and the input from the pulse receiver 430 are accumulated by the beamforming receiver 432. Then a signal processor 434 processes and analyzes the array of digital signals. The signal processor 434 may be wirelessly connected 440 to a display device 438 for display of the ultrasound results and ultimate control of the bladder volume measurement device by a user. A power supply 436 may also provide power to the components of the bladder volume measurement device 400 and all of its components. Overall, this bladder volume measurement device 400 may consist of a 2-dimensional (2D) ultrasound sensing array 412 with parallel transmit-receive electronic circuitry, analog-to-digital (A/D) conversion, beamforming, and signal processing before wireless data transmission to the display device 438. A microcontroller (not shown) may include one or more of these components or control the operation of one or more of these components. Many of the components may be combined or placed in different locations of this architectural diagram. Accordingly, FIG. 4 is solely designed to show one embodiment of the present disclosure and is not designed to limit the disclosure to this embodiment.

The ultrasound transducer array 412 may consist of a phased array transducer arranged in a 2-dimensional (2D). As shown in FIG. 3, timed excitation and control of the transducer sensors allow beamsteering in broad volume space. Each transducer unit both transmits and records backscatterd ultrasound signals on receipt. Use of lower frequency ultrasound pulses allow ample penetration in human tissue (center frequency=2 MHz). A transducer aperture size of 5 mm in diameter provides an acceptable compromise between penetration and resolution, both laterally and axially. The distance between neighboring elements in the ultrasound transducer array may be selected to allow beamsteering over a range of 0=±35° without grating lobes being generated. In some embodiments, 64 transducer elements may be used with a square shape and approximately 0.4 mm in size, equally spaced (pitch of 0.5 mm), excited using a short voltage pulse length of 1.5 μs, and have a 50% bandwidth of approximately 1 MHz. The surface of the array may have an acoustic impedance matched to water by applying a thin matching lawyer to maximize transmission of ultrasound from the elements to the patient (quarter wavelength, λ/4˜200 μm). Connections to the individual elements in the transducer are made through individual micro-coaxials soldered directly to a printed circuit board of the transmit/receive circuitry. This unique layout of elements provides a wide-angle field-of-view as well as high resolution.

If multiple transducer arrays are deemed necessary to increase the field-of-view and improve bladder volume measures, a multiplexer will be integrated to switch between these redundant electronic units in the interface device. This N-channel multiplexer may be implemented using high-voltage analog switches. During data acquisition, the relevant transducer is connected to the transmitting and receiving circuits using this circuit, and the remaining transducers may be disconnected. The multiplexing circuit may be controlled by a main microcontroller unit.

The transmitter portion of the transmitter/receiver 410 may be a multi-channel transmitting circuit that will be constructed using open-drain high-voltage field effect transistor circuits (FET) circuits, with the source of the transistors connected to −90 V and the drain connected to the phased array elements. The pulse processor (pulser) 402 may be based upon low-threshold, low-input capacitance N-channel enhancement-mode vertical DMOS FETs excited directly by the interface circuit through the capacitors. Beamsteering may be done by the pulse processor (pulser) 402 by phasing the excitation of the individual transmitting channels with a temporal resolution of 65 μs (for an acquisition depth of 10 cm). The excitation pulse duration will be set to 1.5 μs, equivalent to 3 cycles of a 2 MHz pulse. To ensure an adequate equal signal-to-noise ratio (SNR) in all directions, data acquisition will be automatically repeated more times for larger angles than for smaller angles before the backscattered ultrasound signals are averaged (compounded). The receiver portion of the transmitter/receiver 410 may include double clamping-diodes to protect the input. Then the returning backscattered ultrasound signal will be amplified and frequency limited by a low-noise, operational linear amplifier circuit 420. Signal sampling may be performed by an interface circuit (8 MHz) followed by time-gain compensation 422 at the software level to balance progressive signal loss during abdominal tissue propagation, assumed 1.4 dB cm⁻¹. Multiple pulsing strategies may be used including linear pulsing, nonlinear pulsing, and Doppler pulsing. In some embodiments, the components and software for the bladder volume measurement device may need to adjusted based upon the pulsing strategy used.

A power supply 436 may generate the voltages needed from a lightweight rechargeable lithium-ion polymer battery. During standby, only part of the unit may be powered up, and the remaining parts of the system are turned off. High voltages may be generated by means of special high-voltage DC-DC converters. The ability to be recharged allows the unit to be placed and perform bladder volume measurements untethered to a physical charger. Using sensing technology to detect ureter jet actions may assist with accuracy of the bladder volume measurement device and assist with power control. Thus, the bladder volume measurement device may only turn on most of the components after jet action was sensed in the ureter. This type of sensor may also track time and frequency information related to urine traveling from the kidneys to the bladder through the ureter. In additional to bladder volume measurements, jet actions from the ureter and corresponding volume measurements may be analyzed for health status implications.

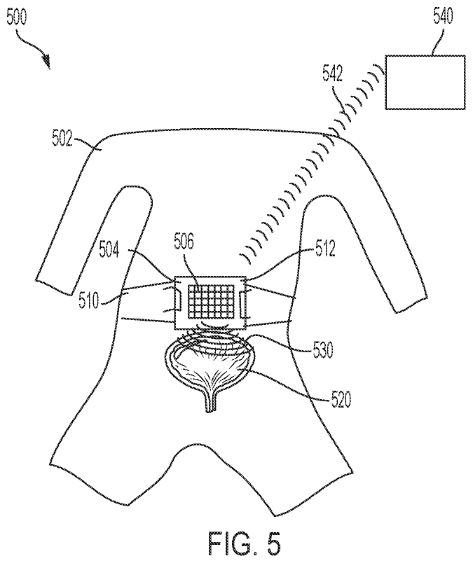

FIG. 5 shows an anterior view 500 of a bladder volume measurement device 512 attached to a patient 502 according to certain embodiments of the present disclosure. FIG. 5 is merely a representation of the device 512 and may not be drawn to scale and may not be located in the proper position on the patient. As discussed above, the patient 502 may be in an operation setting, in the intensive care unit, or in another setting where bladder volume measurements may be required. The bladder volume measurement device 512 may include an ultrasound transducer array 506 as illustrated in FIGS. 1 and 4. In some embodiments, a housing may be located on top of the ultrasound transducer array 506 to or may be located around the ultrasound transducer array 506 to house circuitry, power supply, and the user interface for the bladder volume measurement device 512. Buttons, input devices, or screens may also be located on the bladder measurement device 512 to enable user input and user interaction. An elastic band 510 may be used to hold the bladder volume measurement device 512 in the proper position on the abdomen of the patient 502. Clips or edges 504 may be included to enable the patient 502 or a healthcare provider to clip or slide in the bladder volume measurement device 512. The clips or edges 504 may include an outer region that surrounds and protects the bladder measurement device 512 but enables the device 512 to touch the patient's skin. The elastic band 510 in conjunction with the clips 504 help the user to ensure that the bladder volume measurement device 512 is in the right position on the abdomen of the patient 502. The bladder volume measurement device 512 may need to be attached to the skin for accurate readings, and all patient hair in the corresponding region may need to be removed. The material used for the bladder volume measurement device 512 and/or ultrasound transducer array 506 may need to be isoechoic to prevent any distortion from the skin to device 512 interface. The bladder volume measurement device 512 may need to be initially placed by nurses after training has taken place related to placement of the device 512. Ideally, patients themselves may also be trained to place the device in the suprapubic region.

Placement of the bladder measurement device is crucial to operation. In some embodiments, displays on a computer, laptop, tablet, or smartphone device may show image slices of the suprapubic region with guidance overlays on the image (in acquisition mode). This way, as the device is acquiring the image of the patient's bladder, it can assist the user or healthcare worked with proper positioning of the device for improved and accurate imaging. Key structures of the patient (trigone pelvic landmark, pubis) may be imaged and displayed on the tablet or smartphone device to optimize placement. An elastic band 510 in combination with clips 504 (harness) may secure this placement and apply further pressure on the skin of the patient for improved imaging. Alternatively, a belt may be used in place of the elastic band 510 to secure placement of the device and apply pressure for improved imaging. For example, a belt with similar clips or edges may be used to secure placement of the device. An adhesive material may also be used in conjunction with the elastic band 510 or belt to adhere the bladder volume measurement device to the patient's skin. In some embodiments, the adhesive material may form a frame around the bladder volume measurement device to adhere the device to the skin but enable contact with the skin where necessary. In other embodiments, the adhesive material may be used in place of the elastic band 510 or belt to anchor the bladder volume measurement device in the proper position. An application of a layer of ultrasonic gel may be applied to the surface of the bladder volume measurement device (near the ultrasound transducer array) before placement by a healthcare provider. Adhesive tapes are used to secure the device just above the pubis bone and then straps, harnesses, or belts may be used to hold the device in place.

A disposable interface between the probe of the device (ultrasound transducer array) and the skin that does not induce an allergic reaction and is suitable to be worn for a significant period of time may be used with the present disclosure. In some embodiments, this material adjacent to the skin may be similar in composition to that of an ostomy wafer, but will need to be compatible with a fixed ultrasound device. This disposable interface may be the only disposable part of the bladder volume measurement device.

In operation, the ultrasound transducer array 506 transmits ultrasound pulses or waves 530 towards a bladder 520 of the patient. The ultrasound pulses or waves 530 are then reflected back towards the ultrasound transducer array 506 for processing. The circuitry within the housing 514 may then process the signals and develop a view of the bladder 520 and begin to track a volume within the bladder 520 (through size measurements of the bladder). Then the resulting data 542 may be transmitted back to a display unit 540 to display the results to a user. In other embodiments, the backscattered ultrasound signals may be transmitted back to the display unit 540 before processing so that the processing can take place at the display unit 540. The display unit 540 may be a smartphone, computer, server, laptop, tablet, or other computing device. Thus, the processing may be done through the smartphone, computer, server, laptop, tablet, or other computing device, which would reduce the circuitry that may be required in the bladder volume measurement device 512 and save battery life.

In some embodiments, the housing 512 will house the ultrasound transducer array 506, electronic circuitry (as shown in FIG. 4) with on/off functionality, and a power supply. The electronic circuitry may include a microcontroller with a processor, memory, and input/output peripheral connectivity. The microcontroller may be programmed to control the entire sensing system and record the backscattered ultrasound signals and process the sampled data to calculate clinical metrics of importance. Wireless connectivity 542 may allow communicating metrics to a host system running dedicated system application software. A universal serial port (USB) may also allow direct access to an external computer for system updates, troubleshooting, and implementation of any technical issues.

In some embodiments, a mobile application or software program may remotely control ultrasound data acquisition. The display device may allow a user to save patient information, setup data acquisition parameters (e.g., interval between measurements, alarm mode), or perform manual bladder measurements. If alarm mode is enabled, the program triggers an alarm whenever the bladder volume is over a preset level. The latest measurement is presented on the display, along with a trend curve, and a graphical representation indicating the position of the device in relation to the bladder.

A novel beamsteering technique may be implemented to maximize the accuracy of bladder volume calculations and sensitivity to change after proper device placement. Computer software may be used to direct ultrasound beamsteering and find (converge on) the maximal bladder diameter as detected from the high amplitude peaks in the backscattered ultrasound signals originating from the anterior and posterior bladder wall, as shown in FIG. 2. This approach may be continuously executed to maintain proper ultrasound beam angulation during repeat data acquisitions. Once the proper beam angle is determined, bladder volume and other metrics may be calculated by the microcontroller unit from the sampled backscattered ultrasound signals. First, these sampled signals are converted to distance units given knowledge of the data sampling rate (fₛ) and speed of sound (c). After automated detection of the bladder walls from the median filtered ultrasound signals (kernel size of 11 samples) using computer software for determining a spatiotemporal intensity threshold, these positions may be scan converted to spatial (x, y, z). coordinates. Position vectors may then be used to estimate bladder volume (assuming organ sphericity). Repeat volume measurements can be averaged to improve precision.

Numerous procedures may be used to confirm the accuracy of the bladder volume measurement device. A bladder scanner phantom test can be used with specific volume measurements to test the device. Bladder scanner phantoms can be constructed from multi-layer materials that mimic the muscle and tissue of the bladder wall and urine. Each tissue and organ are independent and have their own characteristic, ultrasound echogenicity and mechanical properties. To assess any system drift, the system scanner may be set to measure bladder volume every 10 minutes for at least a 24-hour period. Accuracy and precision may be calculated based on knowledge of the true volume. The dynamic performance of the bladder volume device may be assessed by varying the fluid volume in the phantom material using a syringe pump and physical measurement of the true fluid volume changes. A linear regression analysis will be used to assess trends in measured versus true volume measures. Any issues with drift or measurement accuracy may be addressed. An initial benchmark of device accuracy less than 5% could be considered acceptable.

Studies with healthy adult volunteers or patients may also be used to confirm the accuracy of the bladder volume measurement device. Volunteers with no prior genitourinary surgery or urological history may be preferable for testing. For each volunteer, pelvic MRI examinations may be conducted to assess pre-void true bladder volume. The bladder volume measurement device may be fixed in place on the suprapubic region for a specific amount of time. The assumption of voiding efficiency greater than 90% will be in place and the voided volumes of patients may be tracked to assess the accuracy of the device. Collection of urine output through either condom catheters, female urine collection systems, or assessing pad weights may also be used to test the accuracy of the device. Residual urine volume may be assessed by placement of an indwelling urethral catheter or an MRI of the pelvis.

Software programs and/or artificial intelligence may be used to improve the accuracy of bladder volume measurements by predicting volume measurements based on multimodal imaging (liner/nonlinear). More specifically, the software programs may further reject poor images, identify key structures (trigone pelvic landmark, pubis) to better isolate bladder information, predict full contour of the bladder, and average multiple volume captures. Transmission of pulses for traditional brightness-modulated (B-mode) ultrasound imaging, nonlinear imaging, and color Doppler imaging may be used to improve detection of the bladder tissue boundary and detect ureter jets. Both B-mode and nonlinear ultrasound images may be used for bladder edge segmentation analysis. With respect to ureter jet activity, a color Doppler image of ureter jet activity may be overlaid on B-mode ultrasound images and displayed to the user. Additionally, the frequency of ureter jet activity can be measured and displayed.

The design of the bladder measurement device and its corresponding placement on the patient may also be adjusted to improve accuracy in view of patient motion or positioning. For example, sensors may be added to the device to identify when the patient moves or adjusts positioning to account for changes in the reading from the bladder, including the volume measurement readings. In some embodiments, one or more axis accelerometer and altitude detection sensors may be included within the circuitry of the bladder volume measurement device to detect movement of the patient and correspondingly adjust the readings. These features of the present disclosure may assist with obtaining highly accurate results even when the bladder volume measurement device is placed in a suboptimal position by the healthcare provider or user.

Enabling the device to be attached to the patient and be used without the use of wires may be a significant feature in some embodiments of the present disclosure. With a battery as the power supply, lower power during operation becomes important. The device may be enabled to operate in multiple modes that allow lower voltage sensing. Transition to and from these modes may be controlled by circuitry. These lower power modes may include sparse array activation during times of continuous operation. As discussed above, sensors may be used to wake up the device upon jet actions of the ureter or urethra. A number of raw measurements may also be averaged initially to form a measurement output, thereby saving numerous measurement outputs or transmissions.

Complementary devices are within the scope of the present disclosure. For example, a urine catchment system may be included in the bladder volume measurement device to remove the expelled urine from around the patient's body into a secondary storage device below the patient. This may include a negative pressure system that will be applied to a device that is placed around the patient's pelvis for collecting any expelled liquids. In some embodiments, the volumes of expelled liquids could be used to further confirm the accuracy of the bladder volume measurement device. In some embodiments, one or more pressure sensors could provide synchronous pressure measurements from the bladder to identify underlying pathological disorders of the lower urinary tract system. The bladder volume measurements may be used in conjunction with the pressure readings to analyze the patient's condition.

In addition to the advantages discussed above, the present disclosure has the ability to identify patients at risk for urinary retention and may be able to determine which patients warrant indwelling urethral catheterization. Post-operative urinary retention is a binary condition, however, this bladder volume measurement device may be able to define a patient's voiding efficiency (voiding efficiency=[voided volume/total bladder capacity]×100) as a continuous variable, and may serve as a clinical tool to identify which patients are at risk for recurrent infections and renal insufficiency secondary urinary retention.

Aspects disclosed herein include:
A. A medical device for determining a volume of liquid in a bladder of a patient, the medical device including: 1) a pulser that is configured for generating phased electrical pulses; 2) an ultrasound transducer array that is configured to be coupled to said pulser comprising at least two transducer elements, wherein each transducer element is configured to transmit said phased electrical pulses towards a patient's bladder and is configured to receive backscattered electrical pulses that are reflected from said patient; 3) amplifier that is configured to be coupled to said ultrasound transducer array for adjusting said backscattered electrical pulses; 4) a signal processor that is configured for processing said adjusted backscattered electric pulses to track a change in volume of said patient's bladder; and 5) a transmitter that is coupled to said signal processor and is configured to transmit said change in volume of said patient's bladder to a display device.
B. A system for determining a volume of liquid in a bladder of a patient, the system including: 1) a means for generating phased electrical pulses; 2) an ultrasound transducer array that is configured to be coupled to said means for generating comprising a number of transducer elements, wherein each transducer element is configured to transmit said phased electrical pulses towards a patient's bladder and is configured to receive backscattered electrical pulses that are reflected from said patient's bladder; 3) means for amplifying that is configured to be coupled to said ultrasound transducer array for adjusting said backscattered electrical pulses; 4) a means for processing said adjusted backscattered electric pulses to develop a representation of a bladder of a patient; and 5) a means for transmitting that is coupled to said signal processor and is configured to transmit said processed signals.
C. A medical device for determining a volume of liquid within a bladder of a patient, the medical device including: 1) a pulser circuit that is configured for generating phased electrical pulses; 2) a transducer array that is configured to be coupled to said pulser circuit, wherein at least two transducer elements of said transducer array are configured to transmit ultrasound phased electrical pulses towards a patient's bladder and are configured to receive backscattered electrical pulses from said patient; 3) a signal processor that is configured for processing said backscattered electric pulses to develop a representation of said patient's bladder; and 4) a transmitter that is coupled to said signal processor and is configured to transmit said representation of said patient's bladder to a display device.

Aspects A, B, and C may have one or more of the following additional elements in combination: Element 1: wherein said amplifier further comprises a time gain compensation amplifier. Element 2: wherein said ultrasound transducer array is further configured for beamsteering of said phased electrical pulses. Element 3: wherein said transmitter is further configured to wirelessly transmit said processed backscattered electrical pulses to said display device. Element 4: wherein said ultrasound transducer array is further configured to be adjacent to said patient's skin in a suprapubic region of said patient. Element 5: further comprising a harness for holding said ultrasound transducer array adjacent to said patient's skin in said suprapubic region of said patient. Element 6: further comprising a least one sensor for sensing a movement of said patient, wherein said signal processor is configured to adjust processing of said backscattered electrical pulses in response to said movement. Element 7: wherein said signal processor is further configured to process said backscattered electrical pulses to identify a placement of said ultrasound transducer array on said patient. Element 8: wherein said transmitter is further configured to transmit said position of said ultrasound transducer array to said display device. Element 9: wherein said display device is further configured to provide an alarm signal to a user if said ultrasound transducer array is in an improper position. Element 10: further comprising a means for supporting that is configured to support said ultrasound transducer array to be adjacent to said patient's skin in a suprapubic region of said patient. Element 11: further comprising a sensing means for sensing a movement of said patient, wherein said means for processing adjusts processing of said backscattered electrical pulses in response to said movement. Element 12: wherein said means for processing is further configured to process said backscattered electrical pulses to identify a placement of said ultrasound transducer array on said patient. Element 13: wherein said means for transmitting is further configured to transmit said placement of said ultrasound transducer array to a means for display.

Although the present disclosure and its advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A medical device for determining a volume of liquid in a bladder of a patient, comprising:
a pulser that is configured for generating phased electrical pulses;
an ultrasound transducer array that is configured to be coupled to said pulser comprising at least two transducer elements, wherein each transducer element is configured to transmit said phased electrical pulses towards a patient's bladder and is configured to receive backscattered electrical pulses that are reflected from said patient;
amplifier that is configured to be coupled to said ultrasound transducer array for adjusting said backscattered electrical pulses;
a signal processor that is configured for processing said adjusted backscattered electric pulses to track a change in volume of said patient's bladder; and
a transmitter that is coupled to said signal processor and is configured to transmit said change in volume of said patient's bladder to a display device.

2. The medical device of claim 1 wherein said amplifier further comprises a time gain compensation amplifier.

3. The medical device of claim 1 wherein said ultrasound transducer array is further configured for beamsteering of said phased electrical pulses.

4. The medical device of claim 1 wherein said transmitter is further configured to wirelessly transmit said processed backscattered electrical pulses to said display device.

5. The medical device of claim 1 wherein said ultrasound transducer array is further configured to be adjacent to said patient's skin in a suprapubic region of said patient.

6. The medical device of claim 5 further comprising a harness for holding said ultrasound transducer array adjacent to said patient's skin in said suprapubic region of said patient.

7. The medical device of claim 1 further comprising a least one sensor for sensing a movement of said patient, wherein said signal processor is configured to adjust processing of said backscattered electrical pulses in response to said movement.

8. The medical device of claim 1 wherein said signal processor is further configured to process said backscattered electrical pulses to identify a placement of said ultrasound transducer array on said patient.

9. The medical device of claim 8 wherein said transmitter is further configured to transmit said position of said ultrasound transducer array to said display device.

10. The medical device of claim 8 wherein said display device is further configured to provide an alarm signal to a user if said ultrasound transducer array is in an improper position.

11. A system for determining a volume of liquid in a bladder of a patient, comprising:
a means for generating phased electrical pulses;
an ultrasound transducer array that is configured to be coupled to said means for generating comprising a number of transducer elements, wherein each transducer element is configured to transmit said phased electrical pulses towards a patient's bladder and is configured to receive backscattered electrical pulses that are reflected from said patient's bladder;
means for amplifying that is configured to be coupled to said ultrasound transducer array for adjusting said backscattered electrical pulses;
a means for processing said adjusted backscattered electric pulses to develop a representation of a bladder of a patient; and
a means for transmitting that is coupled to said signal processor and is configured to transmit said processed signals.

12. The system of claim 11 wherein said ultrasound transducer array is further configured for beamsteering of said phased electrical pulses.

13. The system of claim 12 further comprising a means for supporting that is configured to support said ultrasound transducer array to be adjacent to said patient's skin in a suprapubic region of said patient.

14. The system of claim 11 further comprising a sensing means for sensing a movement of said patient, wherein said means for processing adjusts processing of said backscattered electrical pulses in response to said movement.

15. The system of claim 11 wherein said means for processing is further configured to process said backscattered electrical pulses to identify a placement of said ultrasound transducer array on said patient.

16. The system of claim 15 wherein said means for transmitting is further configured to transmit said placement of said ultrasound transducer array to a means for display.

17. A medical device for determining a volume of liquid within a bladder of a patient, comprising:
a pulser circuit that is configured for generating phased electrical pulses;
a transducer array that is configured to be coupled to said pulser circuit, wherein at least two transducer elements of said transducer array are configured to transmit ultrasound phased electrical pulses towards a patient's bladder and are configured to receive backscattered electrical pulses from said patient;
a signal processor that is configured for processing said backscattered electric pulses to develop a representation of said patient's bladder; and
a transmitter that is coupled to said signal processor and is configured to transmit said representation of said patient's bladder to a display device.

18. The medical device of claim 17 wherein said transducer array is further configured for beamsteering of said ultrasound phased electrical pulses.

19. The medical device of claim 17 further comprising at least one sensor for sensing a movement of said patient, wherein said signal processor is further configured to adjust processing of said backscattered electrical pulses in response to said movement.

20. The medical device of claim 17 wherein said signal processor is further configured to process said backscattered electrical pulses to identify a placement of said transducer array on said patient.
